(19) United States
(12) Patent Application Publication
(10) Pub. No.: US 2007/0207192 A1
(43) Pub. Date: Sep. 6, 2007

(54) Adhesive bioerodible transmucosal drug delivery system

(75) Inventors: Richard Holl, Park City, UT (US); David W. Osborne, Santa Rosa, CA (US)

Correspondence Address: LAHIVE & COCKFIELD, LLP, ONE POST OFFICE SQUARE, BOSTON, MA, 02109-2127, US

(73) Assignee: QLT USA, Inc., Fort Collins, CO (US)

(21) Appl. No.: 11/645,091

(22) Filed: Dec. 22, 2006

Related U.S. Application Data
(63) Continuation of application No. 11/355,312, filed on Feb. 15, 2006.
Application No. 11/355,312 is a continuation of application No. PCT/US04/26531, filed on Aug. 16, 2004.
(60) Provisional application No. 60/495,356, filed on Aug. 15, 2003.

(51) Int. Cl. A61K 9/70; A61K 36/254; A61K 36/84; A61K 36/539; A61K 36/16; A61K 36/8962; A61K 36/81; A61K 36/28; A61K 36/258

(52) U.S. Cl. 424/449; 424/725; 424/728; 424/764; 424/733; 424/741; 424/760; 424/754; 424/752; 424/769

(57) ABSTRACT

The present invention is directed to a mucoadhesive delivery system for the local or systemic administration of a pharmaceutical agent. The delivery system of the invention effectively and facilely enables transport of the pharmaceutical agent through mucosal membranes and into the vasculattire of the mucosa. The delivery system includes an at least partially water soluble bioadhesive layer and an at least partially water soluble backing layer. Incorporated within either or both of these layers are the pharmaceutical agent and a mucosal penetration enhancing agent. The mucosal penetration enhancing agent displays localized tissue irritation properties. The mucoadhesive delivery system may be in the form of a gel, film, disc or patch. It may be applied to any mucosal membrane of a patient including but not limited to those of the buccal and nasal cavities, throat, eye, vagina, alimentary tract and peritoneum.

### STATEMENT OF PRIORITY

This application is a Continuation Under 35 U.S.C. §1.111(a) of International Application No. PCT/US2004/026531, filed Aug. 16, 2004 and published in English as WO 2005/016321 on Feb. 24, 2005, which claims the benefit under 35 U.S.C. §119(e) of U.S. Provisional Application No. 60/495,356, filed Aug. 15, 2003, which applications are incorporated herein by reference.

### FIELD OF THE INVENTION

The present invention relates generally to bioerodible, water-soluble systems for transmucosal delivery of pharmaceutical agents for either systemic or local therapy.

### BACKGROUND OF THE INVENTION

Several mucoadhesive devices are available for use to deliver pharmaceutical agents locally or systemically through a mucous membrane within the body. Many of these devices are in the forms of films or patches that conveniently fit within a body cavity (e.g., mouth) and adheres to a mucous membrane. They are often designed to be pressure sensitive, and they adhere immediately upon application to membranes.

The BEMA™ (Bioerodible Muco-Adhesive System) Drug Delivery System is a bioerodible film for fast-acting local or systemic delivery of pharmaceutical agents. The BEMA™ technology provides a mucoadhesive and bioerodible disc for application to a mucosal surface and is used for transmucosal delivery of drugs over variable lengths of time, e.g., delivery occurring for minutes or hours. The BEMA technology is disclosed, e.g., in Tapolsky, et al. (U.S. Pat. No. 5,800,832) and Tapolsky, et al. (U.S. Pat. No. 6,159,498).

Absorption of pharmaceutical compounds through the mucosa is often hampered by the mucopolysaccharide structure of the mucosa, its mucin coating and by the flow of fluid from the mucosa. Consequently, researchers have attempted to design formulations that enhance and accelerate absorption of pharmaceutical agents applied to the mucosa. Their designs, however, have not met with success. Mucosal fluid flow often tends to interfere with absorption. Absorption enhancing chemicals also are not effective. These chemicals are modeled as dermal absorption enhancers and are required to be non-inflammatory at least and anti-inflammatory at best. These characteristics are said to avoid damaging tissue reddening, inflammation and tissue sloughing.

Accordingly, what is needed is a system for facile transmucosal delivery of pharmaceutical agents for either systemic or local therapy, over variable lengths of time, e.g., delivery occurring for minutes or hours. The system would preferably be in the form that would conveniently adhere to a mucosal surface. The system would preferably have suitable bioadhesive capability, such that it would adhere immediately upon application to a mucosal surface. Additionally, the pharmaceutical agent within the system would be effectively transported across the mucosa. These features will maintain and deliver the pharmaceutical agent at the site of treatment for an effective period of time. The system would preferably be bioerodible and biodegradable.

### SUMMARY OF THE INVENTION

The present invention is directed to a bioerodible, at least partially water-soluble delivery system for transmucosal delivery of pharmaceutical agents for either systemic or local therapy, over variable lengths of time, e.g., delivery occurring for minutes or hours. The delivery system is in the form of a gel, system or patch that conveniently fits on or otherwise adheres to a mucosal surface. The system is pressure sensitive and has suitable bioadhesive capability, such that it adheres immediately upon application to a mucosal surface. The system maintains intimate contact with the mucosal surface to achieve rapid onset of therapeutic effects. The system of the present invention creates intimate contact so that the interface of the mucosal surface and system surface is not easily displaced.

The delivery system of the present invention maintains the pharmaceutical agent at the site of treatment for an effective period of time and facilitates agent transport through the action of a mucosal penetration enhancing agent. The penetration enhancing agent is exclusively adapted to operate upon the mucosa as opposed to the skin. For this reason, the delivery system of the present invention has advantages compared to other known mucosal drug delivery systems. The mucosal penetration enhancing agent, at least in part, enables rapid transport of the pharmaceutical agent across the mucosal surface so that mucosal fluids such as saliva, mucin and vaginal fluid do not redirect the transport of the pharmaceutical agent by removing it from the surface of the system.

The mucoadhesive delivery system of the present invention can be placed on any mucosal surface including buccal, vaginal, nasal, rectal, eye, alimentary and peritoneal surfaces. The choice of mucosal surface can be determined in part by the treatment regimen sought.

The mucoadhesive delivery system of the present invention is composed of several components including an at least partially water-soluble bioadhesive layer, an at least partially water-soluble, non-adhesive backing layer, at least one pharmaceutical agent and at least one mucosal penetration enhancing agent. The bioadhesive layer includes at least one bioadhesive polymer and optionally at least one first film-forming, at least partially water-soluble, polymer. The water-soluble non-adhesive backing layer includes at least one second, at least partially water-soluble, film-forming polymer. The pharmaceutical agent may be one or more pharmaceutical compounds and may be distributed within the bioadhesive layer, distributed within the non-adhesive layer, or distributed within both. The mucosal penetration enhancing agent may be in admixture with the pharmaceutical agent wherever the agent is located or may be found only in the bioadhesive layer. The mucoadhesive delivery system is compatible with mucosal surfaces. It adheres to mucosal surfaces. It is flexible, water-soluble, biodegradable, and bioerodibie. The mucoadhesive delivery system of the invention may be in the form of a flowable gel, a film, a patch or other shaped solid or semi-solid form.

Optional additional layers may also form part of the mucoadhesive system of the present invention. At third layer designed to affect the degradation and release kinetics of the system may be included. A third layer designed to function as a lubrication layer may also be included. The additional layer or layers may also be in the form of coatings applied to the bioadhesive layer, the backing layer or both. The coating can be formed of the same materials as optional third layers and can serve the same purpose or purposes.

The present invention also is directed to methods for treatment using the above-described delivery system. In one embodiment, the method involves delivering a pharmaceutical agent to a mucosal surface of a mammal by contacting the mucosal surface of the mammal with a mucoadhesive delivery system of the present invention and allowing the delivery system to transport the pharmaceutical agent into the blood stream by passage through the mucosa. The delivery kinetics and efficiency are improved over known mucoadhesive devices. The improvements are the result of the presence of the mucosal penetration enhancing agent.

The present invention also is directed to a method for treating a wound on an mucosal surface of a mammal by contacting the mucosal surface of the mammal afflicted with the wound with a mucoadhesive delivery system of the present invention. The system would contain appropriate antibiotics and optional tissue growth hormones.

The present invention also provides a method for locally delivering one or more pharmaceutical agents to a mucosal region of a mammal. In this method, the pharmaceutical agents remain localized throughout the chosen mucosal region. The penetration enhancer enables thorough dispersal of the pharmaceutical agent throughout the mucosal tissue. A rapid dissolution of the delivery system enables local administration while largely avoiding systemic distribution. One embodiment of this method is the use of the delivery system to provide spermicidal or antiviral compounds to the vaginal tract.

Another embodiment is a method for treatment involving the application of a two layer gel to an internal mucosal surface such as that of the large or small intestine or the peritoneum. Use of a two channel delivery apparatus can provide the desired delivery of the flowable two layer gel.

The medical therapies for which use of the mucoadhesive system is appropriate include transmucosal delivery of lipophilic and polar pharmaceutical agents, treatment of pain, cancer and/or dermatological disorders and local or systemic delivery of pharmaceutical agents.

The present invention also is directed to a kit that includes the mucoadhesive delivery system of the present invention and instructions for its use.

### DEFINITIONS

As used herein, certain terms have the following meanings. All other terms and phrases used in this specification have their ordinary meanings as one of skill would understand. Such ordinary meanings may be obtained by reference to such technical dictionaries as Hawley's Condensed Chemical Dictionary 11^{th} Edition, by Sax and Lewis, Van Nostrand Reinhold, New York, N.Y., 1987; The Merck Index, 11^{th} Edition, Merck & Co., Rahway N.J. 1989; The Physician's Desk Reference (PDR), 2001 Edition, Medical Economics Company, Montvale, N.J.; Stedman's Medical Dictionary, 25^{th} Edition, Williams & Wilkens, Baltimore, Md., 1990; and by reference to an English dictionary especially such as “Webster's New World Dictionary of the American Language” College Edition, The World Publishing Co. Cleveland, Ohio and New York, N.Y, 1962.

In the context of the present invention, the term “mucosal” or “mucosa” refers to the mucous membranes of the buccal cavity, nasal cavity, rectum, vagina, urethra, throat, alimentary canal, peritoneum and eye. The mucosa of the stomach, small and large intestine and peritoneum are included as tissues for contact with a mucoadhesive delivery system specially designed to provide adherence to these tissues. Such designs include but are not limited to capsules designed to release the mucoadhesive system upon contacting that specific tissue, and to two layer gels.

In the context of the present invention, the term “mucous” or “mucosal fluid” has its ordinary meaning including but not limited to the secretions of any mucosa and may contain mucins as that term is defined in “The Merck Index” 11^{th} edition, 1989, item no. 6207.

In the context of the present invention, the term “mucopolysaccharides” has its ordinary meaning including but not limited to the structural polymers of the mucous membranes as that term is defined in “Hawley's Condensed Chemical Dictionary” 11^{th} edition, VanNostrand Reinhold Company, New York, N.Y., 1987.

In the context of the present invention, the term “vasculature” refers to the distribution of blood vessels in an organ or tissue.

In the context of the present invention, the terms “at least partially water soluble” and “water soluble” mean that the substance described exhibits a water solubility ranging from negligible to completely water soluble. The substance may readily dissolve in water or may only partially dissolve in water with difficulty over a long period of time. Furthermore, the substance may exhibit a differing solubility in body fluids compared with water because of the complex nature of body fluids. For example, a substance that is negligibly soluble in water may show a solubility in body fluids that is slight to moderate. However, in other instances, the solubilities of a substance in water and body fluid may be approximately the same.

In the context of the present invention, the term “water-soluble polymer” means that the polymer is water swellable and will form a dispersion with water. Depending upon the concentration of the polymer in the water, the resulting dispersion will have a viscosity ranging from fluid like water to viscous or gel-like. The water-soluble polymer will also be water erodible. A water-soluble polymer, however, does not dissolve in water like sodium chloride dissolves in water to form a solution of ions.

In the context of the present invention, the phrase “at least one” means that one or a multiple number of species falling with in the specified generic class can be present. For example, the phrase “at least one bioadhesive polymer” present in the bioadhesive layer means that this layer may be formed of one or a multiple number of bioadhesive polymers. Similarly, the phrase “at least one pharmaceutical agent” means that one or a multiple number of pharmaceutical agents may be present.

### DETAILED DESCRIPTION OF THE INVENTION

The present invention provides a mucoadhesive delivery system that includes a water-soluble bioadhesive layer, a water-soluble non-adhesive backing layer, a pharmaceutical agent and a mucosal penetration enhancing agent. The mucoadhesive delivery system is an extended release delivery system for local and/or systemic administration of pharmaceutical agents. The kinetics and efficiency of delivery are improved relative to the delivery abilities of known mucoadhesive delivery systems. The presence of a mucosal penetration enhancing agent accomplishes this improved ability.

Since the mucoadhesive delivery system can be placed on any internal or exposed mucosal surface, the mucoadhesive delivery system is formulated with pharmaceutically acceptable materials in its bioadhesive layer, its backing layer and as its pharmaceutical agent and penetration enhancing agent. Where appropriate and if desirable, some of these components can be formulated with materials generally regarded as safe (“GRAS-certified), or national formulary certified (“NF-certified”).

Function of the Delivery System

Delivery of active agents (the pharmaceutical agent and penetration enhancing agent) from the delivery system to the surface of the mucosa facilitates the effective function of the delivery system according to the invention. Although the invention is not intended to be limited by the theories concerning this delivery, it is believed that the delivery is accomplished by one or more actions including diffusion, biodegradation, bioerosion and microcapillary activity. Through these delivery actions, the active agents come into contact with the mucosa where the enhanced transport of the pharmaceutical agent through the agency of the penetration enhancing agent is accomplished.

The diffusion mechanism is believed to cause transport of these agents from a high concentration at the interior of the system to a lower concentration at the interface between the system and the mucosa. The biodegradation mechanism is believed to remove the surface layer of the system by degradation of the system polymers thus exposing new surface and the pharmaceutical agent and enhancing agent present there. The bioerosion mechanism is believed to solubilize or otherwise dissolve the polymers at the system surface which also exposes new system surface. The microcapillary mechanism is believed to cause movement of the active agents through micropores and channels present in the delivery system layers. This movement is regarded as distinct from diffusion since it follows microchannels around solid material rather than diffusing through such solid material. All of these mechanisms deliver the active agents to the interface where they can begin their transport into the mucosa.

The mucosa, however, is an active surface. Mucous, saliva, and other body secretions such as vaginal fluid form a fluid flow over, under and around the system surface. This fluid flow has a tendency to misdirect or otherwise remove the pharmaceutical agent and penetration enhancing agent from the interface and dispose of it externally or through the alimentary canal. Additionally, the mucosal surface and mucopolysaccharides are adapted to prevent absorption of polar compounds, ionic compounds and any other compounds not subject to an active absorption pathway through the mucosa. Consequently, many pharmaceutical agents do not penetrate or transport well across the mucosal surface.

The present invention solves this problem presented by the mucosa. The present invention achieves effective penetration of pharmaceutical agents through the action of the penetration enhancing agent.

The character of the mucosal penetration enhancing agent used according to the present invention is surprising in the context of a topical delivery system. The mucosa is commonly considered to be a part of the epidermal system that includes skin. These epidermal layers are regarded as having common functions and properties. They resist or prevent absorption of exogenously applied chemicals and compositions. They are lipophilic in nature. They repel aqueous and organic media and liquids. Consequently, topical formulations are described as being useful on the skin and mucosa. One of skill understands that the positive and negative properties of skin penetration enhancers are also appropriate properties of mucosal penetration enhancers. For example, in U.S. Pat. No. 6,299,900, it is said that the claimed penetration enhancer is effective for transport of a drug across the skin or mucosa. According to this patent, a key negative feature disqualifying a compound as an effective penetration enhancer is its ability to cause irritation. Consequently, current understanding in this field indicates that irritants cannot be used to enhance penetration of the skin or mucosa.

It is surprising, therefore, that appropriate, effective amounts of irritants act as effective mucosal penetration enhancers according to the present invention. Although the invention is not to be limited by a mechanism of action, it is believed that mucosal irritants act by causing local dilation of the capillaries of the mucosa. The irritants are believed to also cause a rapid influx of interstitial fluid within the local mucosa. It is believed that the capillary dilation and increased flow of interstitial fluid enable rapid uptake of the pharmaceutical agent being delivered to the mucosal surface. It is also believed that the penetration of the irritant through the mucopolysaccharide structure of the mucosa facilitates transport of the pharmaceutical agent to these dilated capillaries and the interstitial fluid. It has also been found that the degree of irritation and degree of penetration do not correlate so that the amount of mucosal penetration enhancing agent needed to enable mucosal transport of the pharmaceutical agent usually will not cause significant mucosal irritation. Nevertheless, some degree of irritation can be tolerated according to the invention.

Structure of the Components of the Delivery System

Water-soluble Bioadhesive Layer

The water-soluble bioadhesive layer can adhere to the mucosal surface of any mucosal membrane of a mammal. The water-soluble bioadhesive layer is generally water-soluble and can be made from a bioadhesive polymer(s) and optionally, a first film-forming water-soluble polymer(s). The bioadhesive layer will include at least one pharmacologically acceptable polymer known for its bioadhesive capabilities (the “bioadhesive polymer”) and can optionally include at least one first film-forming water-soluble polymer (the “film-forming polymer”). Alternatively, the bioadhesive layer can be formed of a single polymer that acts as both the bioadhesive and the first film-forming polymer. Additionally, the water-soluble bioadhesive layer can include other first film-forming water-soluble polymer(s) and water-soluble plasticizer(s), such as glycerin and/or polyethylene glycol (PEG).

Bioadhesive Polymer

The bioadhesive polymer of the water-soluble bioadhesive layer can be any water soluble substituted cellulosic polymer or substituted olefinic polymer wherein the substituents may be ionic or hydrogen bonding, such as carboxylic acid groups, hydroxyl alkyl groups, amine groups and amide groups. For hydroxyl containing cellulosic polymers, a combination of alkyl and hydroxyalkyl groups will be preferred for provision of the bioadhesive character and the ratio of these two groups will have an effect upon water swellability and disperability. Examples include polyacrylic acid (PAA), which can optionally be partially crosslinked, sodium carboxymethyl cellulose (NaCMC), moderately to highly substituted hydroxypropylmethyl cellulose (HPMC), polyvinylpyrrolidone (PVP, which can optionally be partially crosslinked), moderately to highly substituted hydroxyethylmethyl cellulose (HEMC) or combinations thereof. In one embodiment, HEMC can be used as the bioadhesive polymer and the first film forming polymer as described above for a bioadhesive layer formed of one polymer. These bioadhesive polymers are preferred because they have good and instantaneous mucoadhesive properties in a dry, system state. Other bioadhesive polymers having similarly useful properties and that are known to one of skill in the art can also be used.

The simultaneous use of PAA with some grades of PVP can result in the precipitation of one or both components. This precipitation may not be desirable, especially when attempting to form a homogenous layer. Moreover, such precipitation may slightly alter the overall adhesive properties of the mucoadhesive system. It is appreciated that one of skill in the art can recognize these problems and avoid use of those grades of PVP with PAA.

First Film-forming, Water-soluble Polymer

The first film-forming water-soluble polymer(s) of the bioadhesive layer can be hydroxyalkyl cellulose derivatives and hydroxyalkyl alkyl cellulose derivatives preferably having a ratio of hydroxyalkyl to alkyl groups that effectively promotes hydrogen bonding. Such first film-forming water-soluble polymer(s) can include hydroxyethyl cellulose (HEC), hydroxypropyl cellulose (HPC), hydroxypropylmethyl cellulose (HPMC), hydroxyethylmethyl cellulose (HEMC), or a combination thereof. Preferably, the degree of substitution of these cellulosic polymers will range from low to slightly above moderate. Similar film-forming water-soluble polymer(s) can also be used. The film-forming water-soluble polymer(s) can optionally be crosslinked and/or plasticized in order to alter its dissolution kinetics.

Water-soluble Non-adhesive Backing Layer

The non-adhesive backing layer is also water-soluble and includes a second, water-soluble, film-forming polymer(s). The non-adhesive backing layer will dissolve after application of the mucoadhesive system to a mucosal surface of a mammal. In many applications, the water-soluble non-adhesive backing layer will typically dissolve before the water-soluble bioadhesive layer dissolves.

The water-soluble non-adhesive backing layer protects the water-soluble bioadhesive layer. Dissolution of the water-soluble non-adhesive backing layer primarily controls the residence time of the mucoadhesive system of the present invention after application to the mucosa and promotes unidirectional delivery across the target membrane.

Second Water-soluble, Film Forming, Polymer

The water-soluble non-adhesive backing layer includes a second water-soluble, film-forming polymer(s). These polymers include polyethers and polyalcohols as well as hydrogen bonding cellulosic polymers having either hydroxyalkyl group substitution or hydroxyalkyl group and alkyl group substitution preferably with a moderate to high ratio of hydroxyalkyl to alkyl group. Examples include, but not limited to, hydroxyethyl cellulose (HEC), hydroxypropyl cellulose (HPC), hydroxypropylmethyl cellulose (HPMC), hydroxyethylmethyl cellulose (HEMC), polyvinyl alcohol (PVA), polyethylene glycol (PEG), polyethylene oxide (PEO), ethylene oxide-propylene oxide co-polymers, and combinations thereof. The water-soluble non-adhesive backing layer component can optionally be crosslinked. In one embodiment, the water-soluble non-adhesive backing layer includes hydroxyethyl cellulose and hydroxypropyl cellulose. The water-soluble non-adhesive backing layer can function as a slippery surface, to avoid “double-stick” to mucous membrane surfaces.

Combinations of different polymers or similar polymers with definite molecular weight characteristics can be used in order to achieve preferred film-forming capabilities, mechanical properties, and kinetics of dissolution.

In all instances, the water-soluble-character of the polymer used is as described in the definitions section.

Third layer or coating specialty polymers and non-polymeric materials may also optionally be employed to impart lubrication, additional dissolution protection, drug delivery rate control, and other specialty character to the transmucosal delivery system. These third layer or coating materials can also include a component that acts to adjust the kinetics of the erodability of the mucoadhesive system. Such third layer or coating materials are described in U.S. patent application Ser. No. 09/684,682, filed Oct. 4, 2000, the disclosure of which is incorporated herein by reference. Examples include polylactide, polyglycolide, lactide-glycolide copolymers, poly-e-caprolactone, polyorthoesters, polyanhydrides, ethyl cellulose, vinyl acetate, cellulose, acetate, polyisobutylene, or combinations thereof.

Pharmaceutical Agent

The pharmaceutical agent(s) can be distributed throughout the water-soluble bioadhesive layer, throughout the water-soluble, non-adhesive backing layer, or throughout both layers. The pharmaceutical agent(s) can be distributed uniformly throughout these layers or can be concentrated in a particular layer such as its concentration near the center of the water-soluble bioadhesive layer.

The pharmaceutical agent can be suitable for local delivery to a particular mucosal membrane or region such as the buccal and nasal cavities, throat, vagina, alimentary canal or the peritoneum. Alternatively, the pharmaceutical agent can be suitable for systemic delivery via such mucosal membranes.

The pharmaceutical agent(s) can be incorporated alone into the bioadhesive and/or backing layers of delivery system or can be preformulated as a pharmaceutical composition which can then be incorporated into these layers. The pharmaceutical composition can include one or more pharmaceutical agents as well as optional excipients, diluants, adjuvants, carriers, polymeric and nonpolymeric viscosity-building agents, polymeric and nonpolymeric hydrophilicity agents, combinations thereof and the like. The pharmaceutical agent(s) and/or pharmaceutical composition(s) can be in the form of a liquid, solid, suspension, molten substance or powder substance when deposited onto either layer of the mucoadhesive system. The agent(s) or composition(s) can be deposited onto either layer more than once. For example, the agent or composition can be deposited onto either layer between about 1 to about 10 times. In this instance, the pharmaceutical agent(s) or composition(s) can be added to the layers after the layers are formed or optionally before the layers are combined together. This kind of preparation procedures is known as “post-loading”.

The pharmaceutical agent or composition can also be added to the layer ingredients as they are being combined into a flowable material prior to coating and subsequent drying. The addition of the pharmaceutical agent or composition prior to system formation is known as “preloading”. The pharmaceutical agent or composition can be either dissolved or dispersed in a liquid or gel. The liquid or gel also includes the polymer(s) and other excipients and the like as described above. This liquid or gel can then processed to form an embodiment of the mucoadhesive system.

The pharmaceutical agent can include a single pharmaceutical compound or a combination of pharmaceutical compounds. Examples of categories of pharmaceutical compounds that can be used, either alone or in combination include: adrenergic agent; adrenocortical steroid; adrenocortical suppressant; alcohol deterrent; aldosterone antagonist; amino acid; ammonia detoxicant; anabolic; analeptic; analgesic; androgen; anti-angiogenic; adjunct to anesthesia; anesthetic; anorectic; antagonist; anterior pituitary suppressant; anthelmintic; antiacne agent; anti-adrenergic; anti-allergic; anti-amebic; anti-androgen; anti-anemic; antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic diagnostic aid; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis synergist; thyroid hormone; thyroid inhibitor; thyromimetic; tranquilizer; treatment of amyotrophic laterial sclerosis; treatment of cerebral ischemia; treatment of Paget's disease; treatment of unstable angina; uricosuric; vasoconstrictor; vasodilator; vulnerary; wound healing agent; and xanthine oxidase inhibitor.

Specific pharmaceutical compounds that are examples of the classes of pharmaceutical compounds disclosed above include, but are not limited to, Acebutolol; Acebutolol; Acyclovir; Albuterol; Alfentanil; Almotriptan; Alprazlam; Amiodarone; Amlexanox; Amphotericin B; Anecortave Acetate; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Eletriptan; Enalapril; Enoxaparin; Ephedrine; Epinephrine; Ergotamine; Etomidate; Famotidirie; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Leuprolide; Levorphanol; Lisinopril; Loratadine; Lorazepam; Losartan; Lovastatin; Melatonin; Methyldopa; Methylphenidate; Metoprolol; Midazolam; Mirtazapine; Morphine; Nadolol; Nalbuphine; Naloxone; Naltrexone; Naratriptan; Neostgmine; Nicardipine; Nifedipine; Norepinephrine; Nortriptyline; Octreotide and analogues thereof; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; rhuFab V2; Rofecoxib; Repaglinide; Risperidone; Rizatriptan; Ropinirole; Somatostatin and analogues thereof; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Visudyne; Zaleplon; Zanamivir; Zafirlukast; Zolmitriptan; and Zolpidem.

The amount of pharmaceutical agent to be incorporated into the delivery system of the invention depends on the desired treatment dosage to be administered, although typically, the pharmaceutical agent will be present in about 0.001% to about 50% by weight of the mucoadhesive system, and more specifically between about 0.005 and about 35% by weight.

Mucosal Penetration Enhancing Agent

The mucosal penetration enhancing agent enables facile transport of the pharmaceutical agent across the mucosal membrane and into the vasculature of the mucosa, or enables dispersement of the pharmaceutical agent throughout the mucosal tissue locally. As discussed above, the mucosal penetration enhancing agent is an irritant. It is surprising that an irritant will have produce such a mucosal transport effect since irritants have a tendency to deter transport through the dermis.

According to the invention, the irritant is believed to function as a local vasodilator that causes dilation of the mucosal vasculature and fluid engorgement of the mucosal tissue. The rapid exchange of fluids of the mucosa is believed to enable distribution rather than isolation of the pharmaceutical agent delivered according to the invention.

The mucosal penetration enhancing agent can be any compound that exhibits a local dilatory and engorging effect upon mucosa. A compound that exhibits systemic vasodilatation, however, is not included within the concept of the mucosal penetration enhancing agents according to the invention.

Classes of mucosal penetration enhancing agent include herbal, plant and chemical substances that cause inflammation, irritation and mucosal reddening. These include the active ingredients in peppers, the topical blistering agents, the common “poison plants” such as poison ivy and poison oak and the like. Chemical agents such as methyl salicylate, menthol, eucalyptus oil, oil of wintergreen and capsicum are also included. Terpenes, triterpenes, oxygenated forms thereof as well as essential oils are also included.

Specific compounds that effectively function as mucosal penetration enhancing agents according to the invention include Capsicum frutescens chili, Allium sativum (Garlic), Amoracia rusticana (Horseradish), Achillea millefolium (Yarrow), Berberis vulgaris (Barberry), Cimicifuga racemosa (Black cohosh), Coleus forskholii (Coleus), Coptis spp. (Goldenthread), Crataegus spp. (Hawthorn), Eleutherococcus senticosus (Siberian ginseng), Ginkgo biloba (Ginkgo), Melissa offiicnalis (Lemon Balm), Olea europaea (Olive leaf), Panax ginseng (Chinese Ginseng), Petroselinum crispum (Parsley), Scutellaria baicalensis (Baical Skullcap), Tilia europaea (Linden Flower), Trigonella foenum-graecum (Fenugreek), Urtica dioica (Nettles), Valeriana officinalis (Valerian), Viburnum spp. (Cramp, Bark, Black Haw), Veratrum viride (American Hellebore), Verbena officinalis (Vervain), Xanthoxylum americanum (Prickly Ash), Zingiber officinale (Ginger) and catechtol derivatives.

As used herein, the term “triterpene” refers to a plant secondary metabolite that includes a hydrocarbon, or its oxygenated analog, that is derived from squalene by a sequence of straightfonvard cyclizations, functionalizations, and sometimes rearrangement. Triterpenes or analogues thereof can be prepared by methods known in the art, i.e., using conventional synthetic techniques or by isolation from plants. Suitable exemplary triterpenes and the biological synthesis of the same are disclosed, e.g., in R. B. Herbert, The Biosynthesis of Secondary Plant Metabolites, 2nd. ed. (London: Chapman 1989), the disclosure of which is incorporated herein by reference. The term “triterpene” refers to one of a class of compounds having approximately 30 carbon atoms and synthesized from six isoprene units in plants and other organisms. Triterpenes consist of carbon, hydrogen, and optionally oxygen. Most triterpenes are secondary metabolites in plants. Most, but not all, triterpenes are pentacyclic. Examples include menthol, eucalyptol, D-limonene, and cymene.

The term, “essential oil” refers to a highly odoriferous, volatile liquid component obtained from plant tissue. Essential oils typically include a mixture of one or more terpenes, esters, aldehydes, ketones, alcohols, phenols, and/or oxides. These functional classes of compounds are responsible for the therapeutic properties and distinct fragrance of the essential oil.

The essential oil can be manufactured (i.e., synthesized or partially synthesized). Alternatively, the essential oil can be obtained from a plant or plant component (e.g., plant tissue). Suitable plant or plant components include, e.g., a herb, flower, fruit, seed, bark, stem, root, needle, bulb, berry, rhizome, rootstock, leaf, or a combination thereof.

Suitable specific essential oils include, e.g., one or more of the following: ajowan, sweet almond oil, allspice, aloe vera oil, ammi visnaga (khella), amyris, angelica root, angelica seed, anise, anise seed, star anise, apricot kernel oil, absolute arnica, avocado oil, unrefined avocado oil, Copaiba balsam, balsam Peru genuine, balsam Peru oil, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot oil, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant),. castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel oil, clove, clove bud, clove leaf, cocoa, cocoa butter, unrefined cocoa butter, coconut oil, refined coconut oil, cognac, combava petitgrain, coriander, green coriander, cornmint, costus oil, cumin, cypress, davana oil, dill, dill weed, elemi, erigeron (fleabane), eucalyptus citriodora, eucalyptus globulus, lemon eucalyptus, fennel, sweet fennel, fenu greek, fir, Canada fir needle, Siberia fir needle, white fir needle, frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed oil, hazelnut oil, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper berry, Siberia juniper berry, Croatia juniper berry, lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, olibanum, absolute opopanax, bitter orange, blood orange, sweet orange, wild West Indian orange, oregano, orris root, concrete orris, osmanthus, palm oil, refined palm oil, palmarosa, paprika, parsley seed, patchouli, Indian patchouli oil, Indonesian patchouli oil, peanut, peanut oil, pecan oil, pennyroyal, pepper, black pepper, super black pepper, peppermint, India peppermint, USA baby mint peppermint, pet perfume, petitgrain (orange leaves), white pine, pine needle, evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthom berry, sesame oil, sesame seed oil, shea butter, unrefined shea butter, spikenard, green spikenard, spruce, St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra.

Other suitable essential oils that can be employed in the compositions of the present invention are disclosed in handbooks such as “CRC Handbook of Terpeniods: Acylic, Monocyclic, Bicyclic, Tricyclic, Tetracyclic, and Pentacyclic Terpenoids, Sukh Dev, Ed., CRC Press, New York N.Y., Cleveland Ohio., 1980-1986, “The Illustrated Encyclopedia of Essential Oils, The Complete Guide to the Use of Oils in Aroma Therapy and Herbalism”, Julia Lawless, Element Books, Ltd., London, UK 1995 and “The Complete Book of Essential Oils and Aroma Therapy”, Valerie Ann Werwood, New World Library, New York, N.Y. 1991 the disclosures of which are incorporated herein by reference.

A non-water soluble lubrication layer can optionally be applied to the water-soluble, non-adhesive backing layer. This would be in the form of a non-continuous system of a silicon or hydrocarbon such as petrolatum. This lubrication layer would provide improved comfort until the delivery system fully hydrates.

Cross-linking Agent

In order to modify the water dissolution kinetics of the backing and/or bioadhesive layers without resulting in a non-water soluble system, limited crosslinking of the layer polymers an be used. When employed, the cross-linking agent will effectively decrease the disintegration rate and lengthen the residence time of the mucoadhesive system. Crosslinking agents known in the art are appropriate for use in the invention and can include, e.g., glyoxal, propylene glycol, glycerol, dihydroxy-polyethylene glycol of different sizes, and butylene glycol. Depending on the particular polymers and crosslinking agent employed, the amount of crosslinking agent can vary, but should not exceed 5% molar equivalent of the bioadhesive polymer and/or the first and/or second water-soluble, film-forming polymer(s), and preferably includes 0% to about 3% molar equivalent of the bioadhesive polymer and/or the first and/or second polymer(s). This limited degree of crosslinking functions to render the bioadhesive polymer and/or the first and/or second film-forming polymers less water soluble. However, the limited degree of crosslinking is not sufficient to render these polymers water insoluble. Typically, these polymers having limited crosslinking remain water swellable and will eventually dissolve or erode in an aqueous medium. The rate of dissolution of the mucoadhesive delivery system can be adjusted by adjusting the degree of limited crosslinking of the bioadhesive polymer and/or the film-forming polymers. Adjusting the rate of dissolution will enable modification of the residence time and the release profile of a pharmaceutical agent(s) within the mucoadhesive delivery system. The limited crosslinking can be included within the bioadhesive polymer and/or the film-forming polymers of the bioadhesive layer or film-forming polymer of the non-adhesive backing layer or both. Selection of one or both layers for inclusion of the limited crosslinking will also affect the dissolution rate of the delivery system.

Physical Dimension

The thickness of the mucoadhesive system of the present invention, in its form as a solid film and the like, may vary, depending on the thickness of each of the layers. Typically, the bilayer thickness ranges from about 0.01 mm to about 1 mm, and more specifically, from about 0.05 mm to about 0.5 mm. The thickness of each layer can vary from about 10% to about 90% of the overall thickness of the bilayer mucoadhesive system, and specifically can vary from about 30% to about 60% of the overall thickness of the bilayer mucoadhesive system. Thus, the preferred thickness of each layer can vary from about 0.005 mm to about 1.0 mm, and more specifically from about 0.01 mm to about 0.5 mm.

When the mucoadhesive system of the present invention is in the form of a gel, the gel layers can be adapted to provide any appropriate thickness. Typically, the gel will be spread as a thin layer over the selected mucosal membrane with the bioadhesive layer being equal to or thicker than the backing layer.

Additives

The mucoadhesive system can also optionally include a pharmaceutically acceptable dissolution-rate-modifying agent, a pharmaceutically acceptable disintegration aid (e.g., polyethylene glycol, dextran, polycarbophil, carboxymethyl cellulose, or poloxamers), pharmaceutically acceptable plasticizer, pharmaceutically acceptable coloring agent (e.g., FD&C Blue #1), pharmaceutically acceptable opacifier (e.g., titanium dioxide), pharmaceutically acceptable anti-oxidant (e.g., tocopherol acetate), pharmaceutically acceptable system forming enhancer (e.g., polyvinyl alcohol or polyvinyl pyrrolidone), pharmaceutically acceptable preservative, or a combination thereof. Preferably, these components are individually present at no more than about 1% of the final weight of the mucoadhesive system, but the amount may vary depending on the pharmaceutical agent(s) or other components of the mucoadhesive system. One of skill in the art can readily determine appropriate concentrations of these components. Several of the preferred additives are discussed individually below.

Plasticizer

The mucoadhesive system can optionally include one or more plasticizers, to soften, increase the toughness, increase the flexibility, improve the molding properties, and/or otherwise modify the properties of the mucoadhesive system. Plasticizers for use in the present invention can include, e.g., those plasticizers having a relatively low volatility such as glycerin, propylene glycol, sorbitol, ethylene glycol, diethylene glycol, triethylene glycol, propylene glycol, polypropylene glycol, dipropylene glycol, butylene glycol, diglycerol, polyethylene glycol (e.g., low molecular weight PEG's), oleyl alcohol, cetyl alcohol, cetostearyl alcohol, and other pharmaceutical-grade alcohols and diols having boiling points above about 100° C. at standard atmospheric pressure (1 atm.). Additional plasticizers include, e.g., polysorbate 80, triethyl titrate, acetyl triethyl titrate, and tributyl titrate. Additional suitable plasticizers include, e.g., diethyl phthalate, butyl phthalyl butyl glycolate, glycerin triacetin, and tributyrin. Additional suitable plasticizers include, e.g., pharmaceutical agent grade hydrocarbons such as mineral oil (e.g., light mineral oil) and petrolatum. Further suitable plasticizers include, e.g., triglycerides such as medium-chain triglyceride, soybean oil, safflower oil, peanut oil, and other pharmaceutical agent grade triglycerides, PEGylated triglycerides such as Labrifil®, Labrasol® and PEG-4 beeswax, lanolin, polyethylene oxide (PEO) and other polyethylene glycols, hydrophobic esters such as ethyl oleate, isopropyl myristate, isopropyl palmitate, cetyl ester wax, glyceryl monolaurate, and glyceryl monostearate. Additional suitable plasticizers include, e.g., those plasticizers disclosed in U.S. Pat. No. 5,700,478.

Disintegration Aid

One or more disintegration aids can optionally be employed to increase the disintegration rate and shorten the residence time of the mucoadhesive system of the present invention. Disintegration aids useful in the present invention include, e.g., hydrophilic compounds such as water, methanol, ethanol, or low alkyl alcohols such as isopropyl alcohol, acetone, methyl ethyl acetone, alone or in combination. Specific disintegration aids include those having less volatility such as glycerin, propylene glycol, and polyethylene glycol.

Dissolution-rate-modifying Agent

One or more dissolution-rate-modifying agents can optionally be employed to decrease the disintegration rate and lengthen the residence time of the mucoadhesive system of the present invention. Dissolution-rate-modifying agents useful in the present invention include, e.g., hydrophobic compounds such as heptane, and dichloroethane, polyalkyl esters of di and tricarboxylic acids such as succinic and citric acid esterified with C6 to C20 alcohols, aromatic esters such as benzyl benzoate, triacetin, propylene carbonate and other hydrophobic compounds as are known in the art. These compounds can be used alone or in combination in the mucoadhesive delivery system of the invention.

Peelable Sheet

In the mucoadhesive system of the present invention, a sheet including the mucoadhesive system may be provided on one side surface and/or the peelable sheet may be provided on one side or both side surface(s), or the sheet may be provided on one side surface and the peelable sheet is provided on another side surface, in view of protection of the adhesive sheet and convenience in handling upon application to human mucosa.

The peelable sheet is not particularly restricted, so long as the sheet is a system having a high peelability. Namely, examples of the system of the peelable sheet include a system including a resin selected from the group polyethylene, polyethyleneterephthalate, polypropylene, polystyrene, polyvinylchloride, polyvinyl alcohol and Saran; polyethylene-coated wood free paper; polyolefin-coated glassine paper; paper, aluminum thin system or the above resins, surface-treated with silicone. Among these, a system including resin of polyethylene or Saran is preferred. The thickness of the peelable sheet can be from about 1 μm to about 500 μm, more specifically from about 5 μm to about 200 μm, and more specifically from about 20 μm to about 100 μm, in viewpoint of handling and cost.

Packaging

The mucoadhesive system of the present invention can be packed in an airtight package system and stored to prevent deterioration in qualities due to moisture. Specific examples of the airtight package system include, e.g., cellophane, moisture proof cellophane, polypropylene, nylon, polyester, vinylidene chloride, vinyl chloride, polycarbonate, low-density polyethylene, high-density polyethylene, linear low-density polyethylene, ionomer, polyvinyl alcohol, ethylene/vinyl acetate copolymer, ethylene/acrylic acid copolymer, ethylene/ethyl acrylate copolymer, polymethylpentene, polystyrene, aluminum foil, etc. Among these systems, systems having polypropylene, vinylidene chloride, low-density polyethylene, high-density polyethylene, linear low-density polyethylene or aluminum foil laminated thereon are particularly preferable due to their excellent barrier properties to vapor permeation. Regarding the barrier properties to vapor permeation, it is preferable that the packed product scarcely suffers from any change in weight when stored at about 40° C./80% relative humidity (RH). The package system for the patch of the present invention preferably results in a weight change of the product of not more than about ±5%, when stored under the above-mentioned conditions for about 6 months.

Preparation

The mucoadhesive system of the present invention can be prepared by numerous methods known in the art. In one embodiment, the components of the separate layers are separately dissolved in the appropriate solvent or combination of solvents to prepare a solution or suspension suitable for coating. Solvents for use in the present invention include, e.g., water, methanol, ethanol, or low alkyl alcohols such as isopropyl alcohol, acetone, methyl ethyl acetone, heptane, or dichloroethane, alone or in combination. The final solvent content or residual solvent content in the system can be the result of either or both layers.

The bioadhesive or backing solutions can then be separately coated onto an appropriate manufacturing substrate. Each solution is cast and processed into a thin system by techniques known in the art, such as by system dipping, system coating, system casting, spin coating, or spray drying using the appropriate substrate. The thin system is then dried. The drying step can be accomplished in any type of oven. However, the drying procedure should be selected to be compatible with the solvent employed and the amount of residual solvent may depend on the drying procedure. One of skill in the art can readily select appropriate drying procedures for the selected solvent(s). The system layers can be prepared independently and then laminated together or can be prepared as systems, one sequentially coated on the top of the other.

The combined system obtained after the layers have been laminated together, or coated on top of each other, can be cut into any type of shape, for application to the tissue. The marginal outline of the mucosal inserts can be triangular, oval circular, ring annular, reniform, square, ellipsoid, bean-shaped, rectangular, or any other symmetrical or unsymmetrical shape.

If the pharmaceutical agent(s) are added to the preformed mucoadhesive system in a liquid form, i.e. postloaded, the solvent used to dissolve or suspend the pharmaceutical agent(s) can vary and typically depends upon the pharmaceutical agent(s) employed, as well as the other components of the mucoadhesive system. Typically, one of skill in the art can select a suitable solvent for the pharmaceutical agent(s) to be incorporated into the mucoadhesive system. Preferred solvents for the composition include organic-based solvents that have a high vapor pressure or a low normal boiling point and that have regulatory acceptance as a pharmaceutical agent solvent suitable for mucosal administration. Examples of solvents that may be used include ethanol or isopropanol.

To postload a mucoadhesive system, an aliquot of the composition solution that includes a therapeutically effective amount of the pharmaceutical agent(s) is applied directly onto the chosen layer of the pre-assembled mucoadhesive system. Preferably, the layer is the bioadhesive layer. Dispensing equipment can be used for applying the pharmaceutical agent composition solution to the selected layer. Examples of microdispensing applicators that can be used include the IVEK® Precision Liquid Metering System. However, any suitable dispensing equipment can be employed. Examples of such dispensing equipment include precision syringes, pipetting equipment, and electronic fluid dispensers.

The aliquot is dried or otherwise stably adsorbed onto the surface of the selected layer to form a pharmaceutical agent-containing deposit on the surface of the mucoadhesive system. Drying of the dispensed solution is by any convenient means known to be acceptable for system drying. Examples of convenient drying methods include drying at ambient conditions or in a conventional system-drying oven. Alternatively, it may be desired for specific product characteristics to maintain the aliquot as a deposit liquid.

The postloaded composition can also be deposited in a solid form. Different solid forms can be used including systems, powders, granules or tablets. The solid form can be prepared by forming a system that contains the pharmaceutical agent(s) and excipients. The system includes water-soluble polymers known to those of skill in the art, for example, some of the water-soluble polymers described herein. Each system can be prepared as a discrete unit, or the system can be divided into discrete units from a larger system, so that the individual systems contain an efficacious amount of the pharmaceutical agent(s). Alternatively, the solid form of the composition can be prepared by compression of a powder mixture using procedures like those used to prepare pharmaceutical agent tablets. Other solid forms of the composition are suitable for application to the mucoadhesive system of the present invention.

The mucoadhesive delivery system can also be preloaded with the pharmaceutical agent or composition. In this preparatory method, the pharmaceutical agent or composition is combined with the layer ingredients and solvent or dispersant. These ingredients are mixed and then processed through the steps described above to form the individual layers and then the bioadhesive delivery system. Appropriate steps are also taken to avoid degradation of the pharmaceutical agent or composition during these processing steps.

The bioadhesive system may also be formulated as a gel. Each layer of the bioadhesive system described above may be combined with a suitable gel forming agent to form the layer into a flowable composition or gel. The gel forming agent may be a pharmaceutically acceptable organic liquid that dissolves or disperses the ingredients of the individual layers and enables these ingredients to be combined and mixed to form a flowable composition. The gel may have a viscosity ranging from low to high as long as it is shapable, moldable or liquid, or in other words, flowable. The agent may be a liquid organic ester, liquid organic amide, liquid organic alcohol, liquid organic acid, liquid hydrocarbon, liquid halogenated hydrocarbon, liquid organic ether, liquid ketone, liquid aromatic compound, and/or a liquid organic amide or a combination thereof. Examples of such organic liquids include triacetin, propylene carbonate, benzyl benzoate, C6 to C20 alcohols, esters of mono-, di- or tri-carboxylic acids and C1 to C20 alcohols, esters of mono-, di- and tri-alcohols and C2 to C20 carboxylic acids, and mono, di and/or triterpenes.

The gel forms of the bioadhesive layer and backing layer can be simultaneously or sequentially applied to a mucosal surface. Once applied, the gels can act directly as the bioadhesive and backing layers or can transform into film layers. The two gel compositions may be separately and sequentially applied to the mucosal surface such that the bioadhesive layer is applied first followed by the backing layer. In another embodiment, both gel compositions may be contained in a dual chamber application device adapted to simultaneously deliver co-joined streams of the gels. The device preferably will indicate the appropriate position of gel application such that the bioadhesive gel will be applied to the mucosal surface and the co-joined backing gel applied on top of the bioadhesive gel. Alternatively, the bioadhesive layer may be used alone.

The gels may be used on any mucosal surface. In particular, the gels may find favorable application to provide irregular and/or extensive coating on such surfaces. In another embodiment, the gels may be used to coat, cover or contact mucosal surfaces that are difficult to reach with non-flowable systems. Such mucosal surface may include vaginal surfaces, nasal surfaces, deep throat surfaces and peritoneal surfaces. A cannula or other system tube or conveyance may be used to deliver the gels to the desired site. Alternatively, the force of expulsion from the gel container chamber may be used to provide deliver to the desired site.

The bioadhesive system may also be formulated to provide its delivery to mucosal membranes not directly accessible by external contact. Such membranes include those of the alimentary canal lining, i.e., the gastrointestinal tract. Formulations for this purpose involve surrounding the bioadhesive system with a protective covering such as a biodegradable capsule designed to disintegrate upon reaching a selected location. The bioadhesive system typically can be preformed as a solid as described above. The solid system is then covered or encapsulated with a coating or a shell that will withstand conditions of certain portions of the gastrointestinal tract but will disintegrate upon contact with other conditions. Use of a polyester coating or capsule shell, such as polylactic acid will enable the delivery of the bioadhesive system to the stomach. Since the polylactic acid disintegrates at low pH, this covering will disintegrate in the stomach. The adhesive side of the bioadhesive system will adhere to the stomach wall where it will deliver the pharmaceutical agent. Similarly, use of a covering such as an enteric coating or enteric capsule shell will permit passage to the small intestine where the higher pH will cause disintegration of the protective covering. The released bioadhesive system will then adhere to the wall of the small intestine and deliver the pharmaceutical agent. Employing a combination of coverings and designing disintegration so as to effect release at a time when gastrointestinal contents reach the large intestine will enable delivery to the large intestine.

Uses of the Mucoadhesive Delivery System

Systems made by the methods of the invention offer the advantages of an effective residence time with minimal discomfort and ease of use, and are an appropriate vehicle for the local as well as systemic delivery of pharmaceutical agent(s), given its flexible form.

Systems formed by the methods of the invention are made of water-soluble components and are bioerodible and biodegradable. The use of water-soluble components allows the mucoadhesive system to dissolve over a period of time, with natural bodily fluids slowly dissolving and eroding away the system, while the pharmaceutical agent(s) remain at the application site. Unlike bandages, transdermal devices and other non-water-soluble system systems, the user of the present invention does not have to remove the mucoadhesive system following treatment. The user experiences minimal sensation of the presence of a foreign object on the mucosal surface, given that upon application, water absorption softens the mucoadhesive system, and over time, the mucoadhesive system slowly dissolves or erodes away.

The residence times of the mucoadhesive systems of the invention depend on the dissolution rate of the water-soluble polymers used. Dissolution rates may be adjusted by mixing together chemically different polymers, such as hydroxyethyl cellulose and hydroxypropyl cellulose; by using different molecular weight grades of the same polymer, such as mixing low and medium molecular weight hydroxyethyl cellulose; by using crosslinking agents such as glyoxal with polymers such as hydroxyethyl cellulose for partial crosslinking; by incorporating hydrophobic agents, such as mineral oil, into the backer formulation; or by post-treatment irradiation or curing, that may alter the physical state of the system, including its crystallinity or phase transition, once obtained. These strategies might be employed alone or in combination in order to modify the dissolution kinetics of the mucoadhesive system, without suppressing the water solubility characteristics of the component systems.

Upon application, the pharmaceutical agent delivery system adheres to the mucosal surface and remains in place. Water absorption softens the mucoadhesive system so that the foreign body sensation is quickly diminished and eliminated. As the system rests upon the mucosal surface, facile delivery of the pharmaceutical agent(s) is enhanced by the action of the mucosal penetration enhancing agent. Residence times may vary, depending on the formulation and systems used, but may be modulated between a few minutes to several hours.

Methods of Treatment Using the Deliverv System of the Invention

In one embodiment, the method of the present invention employs the mucoadhesive delivery system described above to provide local or systemic administration of at least one pharmaceutical agent. The mucoadhesive system is affixed to or otherwise brought into contact with an appropriate mucosal membrane including but not limited to the mucosal membranes of mouth, nasal cavity, vagina, rectum, eye, alimentary canal and peritoneum. Depending upon the selection of pharmaceutical agent and the form of the mucoadhesive system, local or systemic delivery can be achieved. For example, if local delivery is desired, a preferred method would employ a rapidly degrading gel as a coating over the entire mucosal membrane. If systemic delivery is desired, a preferred method would employ a film or patch that does not degrade rapidly. an include an antimigraine medication as the pharmaceutical agent. Any mucosal membrane may be designated as a site for delivery of a pharmaceutical agent to be systemically distributed in the patient. The mucosal membranes located in a patient's head would be preferred delivery sites for treatment of diseases or malconditions of the head and throat. The mucosal membranes of the alimentary tract would be preferred delivery sites for treatment of diseases of the alimentary canal. The mucosal membranes of the peritoneum would be preferred delivery sites for treatment of internal organs. The mucosal membranes of the vagina and/or urethra would be preferred delivery sites for treatment of diseases of the reproductive and urinary tracts and for administration of some forms of antifertility agents.

This method of the invention is appropriate for administration of the classes and species of pharmaceutical agent mentioned in the foregoing section. For example, the buccal and nasal mucosal membranes can be used for delivery of an antimigraine medication contained in the mucoadhesive delivery system of the invention. The mucoadhesive system can be adhered to the buccal mucosa. The antimigraine medication can include, e.g., naratriptan, zolmitriptan, rizatriptan, frovatriptan, octreatide, sumatriptan or other “triptan” pharmaceutical agent. If the mucoadhesive system is placed on the buccal or nasal mucous membranes, it has the advantage of achievement of a rapid plasma level and avoidance of first-pass metabolism.

In another embodiment, the mucoadhesive system of the present invention can include a wound-healing medication as the pharmaceutical agent. The mucoadhesive system would effectively hold the pharmaceutical agent in direct contact with a wound to the mucous membranes such as a wound to vaginal tissue.

In another embodiment, the mucoadhesive system of the present invention can include an antiviral agent, an antibiotic agent, an antifungal agent, a spermicidal agent or a combination thereof The mucoadhesive system would effectively treat infectious diseases (e.g., bacterial, viral, or fungal) or would be a pregnancy preventative agent.

In another embodiment, the mucoadhesive system of the present invention can include an antiviral agent. The mucoadhesive system would deliver the antiviral agent to the tissues involving the mucous membrane of contact, thereby effectively treating patients afflicted with for example venereal herpes.

In a further embodiment, the mucoadhesive system of the present invention can be formulated as a two layer gel as described above. A two channel cannula, application tube, laproscope or other similar two channel application device can be used to apply the gel system can be applied to a selected mucosal membrane. The device would be appropriately oriented so that the bioadhesive gel layer would contact the mucosal membrane and the backing layer would flow over the bioadhesive layer. In one embodiment, the pharmaceutically acceptable solvent in the gel would dissipate leaving a film or coating on the mucosal membrane. In another embodiment, the gel would remain as a gel coating on the mucosal membrane.

### Embodiments

The present invention includes the specific embodiments provided below:
[1] One embodiment of the present invention provides a mucoadhesive delivery system that includes:

a water-soluble bioadhesive layer to be placed in contact with a mucosal surface, the bioadhesive layer including one or more bioadhesive polymers and/or one or more first film-forming, water-soluble polymers;

a water-soluble non-adhesive backing layer that includes one or more second, water-soluble, film-forming, polymers;

one or more pharmaceutical agents distributed within the bioadhesive layer, distributed within the non-adhesive layer, or distributed within both; and

one or more mucosal penetration enhancing agents,

wherein the mucoadhesive delivery system is compatible with mucosal surfaces; adheres to mucosal surfaces; is flexible; and is water-soluble, biodegradable, and bioerodible in mucosal fluids.
[2] Another embodiment of the present invention provides the mucoadhesive system of embodiment [1] wherein the first or second film-forming water-soluble polymer or both includes an alkyl cellulose or a hydroxyalkyl cellulose.
[3] Another embodiment of the present invention provides the mucoadhesive system of embodiment [1] wherein the first film-forming water-soluble polymer includes hydroxyethyl cellulose (HEC), hydroxypropyl cellulose (HPC), hydroxypropylmethyl cellulose (HPMC), hydroxyethylmethyl cellulose (HEMC), or a combination thereof.
[4] Another embodiment of the present invention provides the mucoadhesive system of embodiment [1] wherein the first film-forming, water-soluble polymer includes hydroxypropylmethyl cellulose (HPMC).
[5] Another embodiment of the present invention provides the mucoadhesive system of embodiment [4] wherein the hydroxypropylmethyl cellulose (HPMC) has an average molecular weight (Mw estimated from intrinsic viscosity measurements) in the range about 10² to about 10⁶.
[6] Another embodiment of the present invention provides the mucoadhesive system of any one of embodiments [1]-[5] wherein the first or second or both film-forming water-soluble polymers are cross-linked.
[7] Another embodiment of the present invention provides the mucoadhesive system of any one of embodiments [1]-[6] wherein the first or second or both film-forming water-soluble polymers are plasticized.
[8] Another embodiment of the present invention provides the mucoadhesive system of any one of embodiments [1]-[6] wherein the water-soluble bioadhesive layer is free of a plasticizer.
[9] Another embodiment of the present invention provides the mucoadhesive system of any one of embodiments [1]-[8] wherein the one or more bioadhesive polymers include polyacrylic acid (PAA), sodium carboxymethyl cellulose (NaCMC), polyvinyl pyrrolidone (PVP), or a combination thereof.
[10] Another embodiment of the present invention provides the mucoadhesive system of any one of embodiments [1]-[9] wherein the second water-soluble, film-forming, acceptable polymer includes hydroxyethyl cellulose (HEC), hydroxypropyl cellulose (HPC), hydroxypropylmethyl cellulose (HPMC), hydroxyethylmethyl cellulose (HEMC), polyvinylalcohol (PVA), polyethylene glycol (PEG), polyethylene oxide (PEO), ethylene oxide-propylene oxide co-polymers, or a combination thereof.
[11] Another embodiment of the present invention provides the mucoadhesive system of any one of embodiments [1]-[9] wherein the second water-soluble, film-forming, acceptable polymers include hydroxyethyl cellulose (HEC), hydroxypropyl cellulose (HPC), or a combination thereof.
[12] Another embodiment of the present invention provides the mucoadhesive system of any one of embodiments [1]-[9] wherein the second water-soluble, film-forming, acceptable polymer includes hydroxyethyl cellulose (HEC).
[13] Another embodiment of the present invention provides the mucoadhesive system of embodiment [14] wherein the hydroxyethyl cellulose (HEC) has an average molecular weight (Mw estimated from intrinsic viscosity measurements) in the range about 10² to about 10⁶.
[14] Another embodiment of the present invention provides the mucoadhesive system of any one of embodiments [1]-[13] wherein the water-soluble non-adhesive backing layer further includes a non-water soluble lubrication layer.
[15] Another embodiment of the present invention provides the mucoadhesive system of embodiment [14] wherein the non-water soluble lubrication layer includes an organosilicon-containing compound, a hydrocarbon, or a combination thereof.
[16] Another embodiment of the present invention provides the mucoadhesive system of any one of embodiments [1]-[15] wherein the pharmaceutical agent is selected from the group of adrenergic agent; adrenocortical steroid; adrenocortical suppressant; alcohol deterrent; aldosterone antagonist; amino acid; ammonia detoxicant; anabolic; analeptic; analgesic; androgen; adjunct to anesthesia; anesthetic; anorectic; antagonist; anterior pituitary suppressant; anthelmintic; antiacne agent; anti-adrenergic; anti-allergic; anti-amebic; anti-androgen; anti-anemic; antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic diagnostic aid; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; irnmunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis synergist; thyroid hormone; thyroid inhibitor; thyromimetic; tranquilizer; treatment of amyotrophic laterial sclerosis; treatment of cerebral ischemia; treatment of Paget's disease; treatment of unstable angina; uricosuric; vasoconstrictor; vasodilator; vulnerary; wound healing agent; xanthine oxidase inhibitor; and any combination thereof.
[17] Another embodiment of the present invention provides the mucoadhesive system of any one of embodiments [1]-[16] wherein the pharmaceutical agent is selected from the group of Acebutolol; Acebutolol; Acyclovir; Albuterol; Alfentanil; Almotriptan; Alprazlam; Amiodarone; Amlexanox; Amphotericin B; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Eletriptan; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Leuprolide; Lisinopril; Loratadine; Lorazepam; Losartan; Lovastatin; Melatonin; Methyldopa; Methylphenidate; Metoprolol; Midazolam; Mirtazapine; Morphine; Nadolol; Nalbuphine; Naloxone; Naltrexone; Naratriptan; Neostgmine; Nicardipine; Nifedipine; Norepinephrine; Nortriptyline; Octreotide; Olanzapine; Omeprazole;-Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; Rofecoxib; repaglinide; Risperidone; Rizatriptan; Ropinirole; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Zaleplon; Zanamivir; Zafirlukast; Zolmitriptan; Zolpidem; and any combination thereof.
[18] Another embodiment of the present invention provides the mucoadhesive system of any one of embodiments [1]-[16] wherein the pharmaceutical agent is selected from the group of naratriptan, zolmitriptan, rizatriptan, frovatriptan, sumatriptan, and combinations thereof.
[19] Another embodiment of the present invention provides the mucoadhesive system of any one of embodiments [1]-[16] wherein the one or more pharmaceutical agents are an antiangiogenic agent to the retinochoroid.
[20] Another embodiment of the present invention provides the mucoadhesive system of any one of embodiments [1]-[16] wherein the pharmaceutical agent is an immunosuppressive agent.
[21] Another embodiment of the present invention provides the mucoadhesive system of any one of embodiments [1]-[16] wherein the pharmaceutical agent is an anti-inflammatory agent.
[22] Another embodiment of the present invention provides the mucoadhesive system of any one of embodiments [1]-[16] wherein the pharmaceutical agent is an antibacterial agent.
[23] Another embodiment of the present invention provides the mucoadhesive system of any one of embodiments [1]-[16] wherein the pharmaceutical agent is an antiviral agent.
[24] Another embodiment of the present invention provides the mucoadhesive system of any one of embodiments [1]-[16] wherein the pharmaceutical agent is an antifungal agent.
[25] Another embodiment of the present invention provides the mucoadhesive system of any one of embodiments [1]-[16] wherein the pharmaceutical agent or agents are present in a combined amount of up to about 30 wt. % of the mucoadhesive system.
[26] Another embodiment of the present invention provides the mucoadhesive system of any one of embodiments [1]-[16] wherein the pharmaceutical agent or agents are present in a combined amount of up between about 0.005 wt. % and about 20 wt. % of the mucoadhesive system.
[27] Another embodiment of the present invention provides the mucoadhesive system of any one of embodiments [1]-[16] wherein the pharmaceutical agent or agents are independently located uniformly throughout the bioadhesive layer, uniformly throughout the non-adhesive layer, or uniformly throughout both the bioadhesive and the non-adhesive layers.
[28] Another embodiment of the present invention provides the mucoadhesive system of any one of embodiments [1]-[16] wherein the pharmaceutical agent or agents are independently located uniformly throughout the bioadhesive layer.
[29] Another embodiment of the present invention provides the mucoadhesive system of any one of embodiments [1]-[16] wherein the pharmaceutical agent or agents are independently located near the center of the bioadhesive layer.
[30] Another embodiment of the present invention provides mucoadhesive system of any one of embodiments [1]-[30] wherein the pharmaceutical agent or agents are locally delivered to the mucosal region.
[31] Another embodiment of the present invention provides mucoadhesive systemnof any one of embodiments [1]-[30] wherein the pharmaceutical agent or agents are systemically delivered via the mucosal surface.
[32] Another embodiment of the present invention provides the mucoadhesive system of any one of embodiments [1]-[31] having a thickness of up to about 1 mm.
[33] Another embodiment of the present invention provides the mucoadhesive system of any one of embodiments [1]-[31] having a thickness of between about 0.1 mm to about 0.5 mm.
[34] Another embodiment of the present invention provides the mucoadhesive system of any one of embodiments [1]-[33] further including a pharmaceutically acceptable dissolution-rate-modifying agent, pharmaceutically acceptable disintegration aid, pharmaceutically acceptable plasticizer, pharmaceutically acceptable coloring agent, pharmaceutically acceptable opacifier, pharmaceutically acceptable anti-oxidant, pharmaceutically acceptable system forming enhancer, pharmaceutically acceptable preservative, or a combination thereof.
[35] Another embodiment of the present invention provides the mucoadhesive system of any one of embodiments [1]-[34] wherein the mucosal surface is buccal tissue.
[36] Another embodiment of the present invention provides the mucoadhesive system of any one of embodiments [1]-[34] wherein the mucosal surface is vaginal tissue.
[37] Another embodiment of the present invention provides the mucoadhesive system of any one of embodiments [1]-[34] wherein the mucosal surface is rectum tissue.
[38] Another embodiment of the present invention provides the mucoadhesive system of any one of embodiments [1]-[34] wherein the mucosal surface is nasal cavity tissue.
[39] Another embodiment of the present invention provides the mucoadhesive system of any one of embodiments [1]-[38] further including a third layer located between the water-soluble bioadhesive layer and the water-soluble non-adhesive backing layer; wherein the third layer is flexible, biodegradable, bioerodible in mucosal fluid, and water-soluble.
[40] Another embodiment of the present invention provides the mucoadhesive system of any one of embodiments [1]-[39] further including a component that acts to adjust the kinetics of the erodability of the mucoadhesive system.
[41] Another embodiment of the present invention provides the mucoadhesive system of embodiment [43] wherein the component is a water-based emulsion of polylactide, polyglycolide, lactide-glycolide copolymers, poly-e-caprolactone, polyorthoesters, polyanhydrides, ethyl cellulose, vinyl acetate, cellulose, acetate, polyisobutylene, or combinations thereof.
[42] Another embodiment of the present invention provides a method for treating a wound on an mucosal surface of a mammal including contacting the mucosal surface of the mammal afflicted with the wound, a mucoadhesive system of any one of the foregoing embodiments.
[43] Another embodiment of the present invention provides a method for delivering a pharmaceutical agent to an mucosal surface of a mammal including contacting the mucosal surface of the mammal with a mucoadhesive system of any one of the foregoing embodiments.
[44] Another embodiment of the present invention provides the method of embodiment [43] wherein the delivery of the pharmaceutical agent is local.
[45] Another embodiment of the present invention provides the method of embodiment [43] wherein the delivery of the pharmaceutical agent is systemic.
[46] Another embodiment of the present invention provides the method of any one of foregoing embodiments for use wherein the residence time is up to about 7 days.
[47] Another embodiment of the present invention provides the method of any one of foregoing embodiments for use wherein the residence time is up to about 24 hours.
[48] Another embodiment of the present invention provides the method of any one of embodiments for use wherein the residence time is up to about 8 hours.
[49] Another embodiment of the present invention provides the method of any one of embodiments for use wherein the residence time is between about 1 minute and about 4 hours.
[50] Another embodiment of the present invention provides a method for locally delivering one or more pharmaceutical agents to an mucosal region of a mammal, the method including contacting the mucosal surface of the mammal with a mucoadhesive system of any one of foregoing embodiments.
[51] Another embodiment of the present invention provides a method for systemically delivering one or more pharmaceutical agents to a mammal via an mucosal surface, the method including contacting the mucosal surface of the mammal with a mucoadhesive system of any one of foregoing embodiments.
[52] Another embodiment of the present invention provides a mucoadhesive delivery system as described in any of the foregoing embodiments that is in the form of a flowable gel.

The examples are intended to further illustrate, but not limit, the invention. These examples illustrate compositions for the mucosal delivery of pharmaceutical agents for either local or systemic therapy. The following examples also illustrate the ability of the conjunctiva to provide rapid onset of therapeutic action and increased bioavailability compared to earlier bioadhesive delivery systems.

Those skilled in the art will recognize that, while specific embodiments have been illustrated and described, various modifications and changes may be made without departing from the spirit and scope of the invention.

### EXAMPLE 1

A 200 gm batch of backing stock was manufactured on a weight per weight basis of: 77% purified water, 11% hydroxyethyl cellulose, 11% hydroxypropyl cellulose, and 1% tocopheryl acetate. All systems were mixed until the batch was homogeneous.

A 200 gram batch of water-soluble bioadhesive was made by mixing on a weight per weight basis: 89.5% purified water, 5.5% hydroxypropylmethyl cellulose, 4.4% hydroxyethyl cellulose, 0.5% capsaicin and 0.1 % tocopheryl acetate. Mixing was performed until all components were homogeneous.

### EXAMPLE 2

Using the stock solutions of exampple I,.an Acyclovir bioerodible adhesive drug delivery system can be fabricated. A 6.5% weight per weight basis of Acyclovir can be compounded in the adhesive stock by mixing 9.39 grams of bioadhesive and 0.65 grams of Acyclovir. The stock can be mixed in a Flak Tek mixer for 5 minutes at 3000 rpm, which produced a homogenous solution.

Using a Werner Mathis Labcoater, the substrate, siliconized Mylar, (Rexam 3 mil PET 92A/000), can be secured, and the backing layer solution can be set in front of a knife over-roll with an opening (wet gap) of 0.10 mm. The backing solution can be coated and the system dried for 3.5 minutes at 90° C. The drug loaded bioadhesive can be coated over the dried backer system with a wet gap of 0.50 mm and dried for 5 minutes at 90° C. The bioadhesive system can be cut with a rounded square die cutter (10 mm×10 mm).

A single rounded square Acyclovir delivery system can be placed on the lower gum of a dog with the adhesive side of the bioadhesive system adhered to the mucosal tissue. This can be repeated in five separate dogs with plasma levels of Acyclovir being determined five minutes after application. The plasma levels can be measured in nanograms per milliliter five minutes after administration of the delivery system. Plasma concentrations in the nanograms per milliliter or higher in the dog can be considered to represent therapeutic plasma levels of Acyclovir. Comparison with a bioadhesive system prepared as above but without capsaicin will show that the bioadhesive system of the invention can provide highly effective delivery with the same amount of pharmaceutical agent.

### EXAMPLE 3

Using the stock solutions of example 1, a sumatriptan bioerodible adhesive drug delivery system can be fabricated. A 12% weight per weight basis of sumatriptan succinate can be compounded in the adhesive stock by mixing 17.6 grams of bioadhesive and 2.4 grams sumatriptan succinate. The stock can be mixed in Flak Tek mixer for 5 minutes at 3000 rpm, which produced a homogenous solution.

Using a Werner Mathis Labcoater, the substrate, siliconized Mylar, (Rexam 3 mil PET 92A/000), can be secured, and the backing layer solution can be set in front of a knife over-roll with an opening (wet gap) of 0.10 mm. The backing solution can be then coated and the system dried for 3.5 minutes at 90° C. The bioadhesive with drug can be coated over the dried backer system with a wet gap of 0.50 mm and dried for 5 minutes at 90° C. The bioadhesive system can be cut with a rounded square die cutter (10 mm×10 mm).

### EXAMPLE 4

Using the stock solutions of example 1, a naratriptan bioadhesive drug delivery system can be fabricated. A 7% weight per weight basis of naratriptan hydrochloride can be compounded in the adhesive stock by mixing 18.6 grams of bioadhesive and 1.4 grams of Naratriptan HCl. The stock can be mixed in a Flak Tek mixer for 5 minutes at 3000 rpm, which produced a homogenous solution.

Using a Werner Mathis Labcoater, the substrate, siliconized Mylar, (Rexam 3 mil PET 92A/000), can be secured, and the backing layer solution can be set in front of a knife over-roll with an opening (wet gap) of 0.10 mm. The backing solution can be coated and the system dried for 3.5 minutes at 90° C. The drug loaded bioadhesive can be coated over the dried backer system with a wet gap of 0.50 mm and dried for 5 minutes at 90° C. The BEMA™ system can be cut with a rounded square die cutter (10 mm×10 mm).

Using a similar procedure a control sample of a bioadhesive system containing the drug but not containing capsaisin can be fabricated. The control and experimental samples of the bioadhesive system can be compared by adhering them to wet semipermeable membranes positioned in a vessel. The membranes will separate the container into isolated chambers, the chamber with the bioadhesive system being the system chamber and the other chamber being the transport chamber. The transport chamber can be filled with an isotonic solution. The system chamber can be flushed with a small amount of isotonic solution to simulate fluid flow on the surface of a mucous membrane. After a short period of approximately five minutes, the concentrations of drug in the isotonic solution in each chamber can be measured. It can be determined that the bioadhesive system of the invention is capable of delivering a higher percent of total contained drug to the transport chamber relative to the control. It can also be determined that the bioadhesive system of the invention is capable of avoiding significant flush of the drug into the isotonic solution of the system chamber.

The entire disclosures of all patents, patent-applications, publications and references cited in this specification are incorporated herein by reference as if fully reproduced in this specification.

While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A mucoadhesive delivery system which comprises:
an at least partially water-soluble bioadhesive layer comprising at least one bioadhesive polymer or a combination of at least one bioadhesive polymer and at least one first film-forming, water-soluble polymer;
an at least partially water-soluble non-adhesive backing layer comprising at least one second water-soluble, film-forming polymer;
at least one pharmaceutical agent, and;
a mucosal penetration enhancing agent;
wherein the system is mucoadhesive, is flexible and is biodegradable.

2. A system according to claim 1 wherein the bioadhesive layer comprises more than one bioadhesive polymer alone or in the combination.

3. A system according to claim 1 wherein the backing layer comprises more than one second water-soluble film forming polymer.

4. A system according to claim 1 wherein the bioadhesive layer comprises more than one bioadhesive polymer or a combination of more than one bioadhesive polymer and more than one first film-forming, water-soluble polymer or a combination thereof and the backing layer comprises more than one second water-soluble film-forming polymer.

5. A system according to claim 1 wherein the first film-forming water-soluble polymer comprises an alkyl cellulose or a hydroxyalkyl cellulose.

6. A system according to claim 1 wherein the first film-forming water-soluble polymer comprises hydroxyethyl cellulose (HEC), hydroxypropyl cellulose (HPC), hydroxypropylmethyl cellulose (HPMC), hydroxyethylmethyl cellulose (HEMC), or a combination thereof.

7. A system according to claim 1 wherein the first film-forming, water-soluble polymer comprises hydroxypropylmethyl cellulose (HPMC).

8. A system according to claim 7 wherein the hydroxypropylmethyl cellulose (HPMC) has an estimated average molecular weight in the range about 10² to about 10⁶ based upon intrinsic viscosity measurements.

9. A system of claim 1 wherein the bioadhesive polymer or the first film-forming water-soluble polymer or the second film-forming water-soluble polymer or any combination thereof is cross-linked.

10. A system of claim 1 wherein the first or second film-forming water-soluble polymer is plasticized.

11. A system of claim 1 wherein the water-soluble bioadhesive layer is free of a plasticizer.

12. A system of claim 1 wherein the bioadhesive polymer comprises polyacrylic acid (PAA), sodium carboxymethyl cellulose (NaCMC), polyvinyl pyrrolidone (PVP), or a combination thereof.

13. A system of claim 1 wherein the second water-soluble, film-forming polymer comprises an alkyl cellulose or a hydroxyalkyl cellulose.

14. A system of claim 1 wherein the second water-soluble, film-forming, pharmaceutical agently acceptable polymer comprise hydroxyethyl cellulose (HEC), hydroxypropyl cellulose (HPC), hydroxypropylmethyl cellulose (HPMC), hydroxyethylmethyl cellulose (HEMC), polyvinylalcohol (PVA), polyethylene glycol (PEG), polyethylene oxide (PEO), ethylene oxide-propylene oxide co-polymers, or a combination thereof.

15. A system of claim 1 wherein the second water-soluble, film-forming polymer comprises hydroxyethyl cellulose (HEC), hydroxypropyl cellulose (HPC), or a combination thereof.

16. A system of claim 1 wherein the second water-soluble, film-forming polymers comprises hydroxyethyl cellulose (HEC).

17. A system of claim 13,wherein the hydroxyethyl cellulose (HEC) has an estimated average molecular weight in the range about 10² to about 10⁶ as determined by intrinsic viscosity measurements.

18. A system of claim 1 wherein the water-soluble non-adhesive backing layer further comprises a non-water soluble lubrication layer.

19. A system of 15 wherein the non-water soluble lubrication layer comprises an organosilicon-containing compound, a hydrocarbon, or a combination thereof.

20. A system according to claim 1 wherein the mucosal penetration enhancing agent is able to dilate the vasculature of mucosa.

21-57. (canceled)
